SEQUENCE LISTING (25 sequences)

SEQ ID NO: 1 - DNA, 3844 bp, Artificial Sequence
  Synthesized
atctatgtcgagaggtgcggagatcagcaatggagtggctgatggcacgtgtaatggagtgtgtgagtaactcttcagggttcttgccgaagccctttatataacgttagggcttccggcggcatgcatgtcacaatatcccttataatctgggtacgtgcaatctatttacatgggattggataatatttcttatctcttatcccttcctatacatttttttttgtggggacccttcctatacattaaggatagccgcatggacctggctactcggcctgctctatgtgacacggcacaaaggccttccgtgaggtacccgggtatcgtgccctccacaggtgttgatcgacttttaagtccacagtggtgcaagggatatttcatttactttgagtcgttatatgcatgtggtagcttgatagctacttagttaaagcagtttttatccatatcatctcggaacctccggagaattgactgaaacctaaggttccaaacaattgaccggagactttgagaaataatttaatccgctacaaaagtttgtttaaatttcagttttcgcctattcactcctctctaggctttacaagggactttcagcatccacacaataaaaggtagagttgagcctatcggttatatggagaaaacattaaaaaattgaggatgatgtagagaatataaacaaaagacaaggtagaaactaactgcaagtttatatgcaaatatagcatgtctaattccctcctcatatgtgcaccacacattataatcaacatccaacttggtatgatcatccaatcttggttcgcaaacaccagaagagattgtgcatacatgagcgttttataagtttgtacactaaattacatccaactaacaaattcatatgccatcaatgcaatttgctaacaatgagtttaatagcaaaatgctatactttctatgggtaaattgacactcattttgatgtttgtccattgacaatacaggaaactattctaaactctcgagagaatatcctctcattgtttgcatgtcatccaaatgattataattttctttttaaaaaataagcaagataaattaatatgtgataaatcactccacaagcatgcaaggttaaatttaacttcaacatgttgcaatgagaaaaaaaacaaatttgactatgaatatatgttaactagcttttttttttgcagattgtggaagttaaaatttaacttacatgtttatgcagtgatatatcatatattaatcaattttcttaatttttcataactatttgtatgacatgcaaacaataatgggatatcctttggagagtttaaaatccactccattgacgatatattgtaatactagcataatgctcatgctttgctccatgtaaaggaatatatattatgacagtattatagataaaacatattttttatcgaaaatatatcagtatcacttgtttgcttattatttacatatatggtattcacttatttaaaacgagaaccctagccctagcaaaaccgggccgggctgggccgggcttcactgctttacaagagccagcttagtgaacctaataattctgctcctgtcctccttatcctgatcagtcctcggtcggcttttcctctctctctctctctctctcataaaacagagacctttaatgcgtcgtgatctctcccctccaatcccgacaaagtacacacaccatcgtctccccctcctgcctcgatgctagctctcccattcccatctctccactctccctcctatcatatgctcctcccttctccactcttcttctcaaatcctctctcttcctcttcgtcctcctcctcttgcaaccaaaaaacaccataaaatagtaatcccaatccacaagctcacctctctcttgcacacacacacacctgacacaacacttacacatcataccagaagaaaaaccaattcgattttgccaaattacctagctatagcatataggtaggtagctaggtagcttggttttccctaatagctctagcttgatctctttctagctctaccggcggcgtggcggcggcggcggcggccgcaccactctacatcatcacgtacacctgaccagcttagcgggaagccccacaagaagagtttgtaggtcaccaatcagatcatcagttcatctttgtggttgtgtgggtgtgtgtgtatatatataccatggcgtgagagtagccatggatgtcaccggagacggcggaggaggagggcaacggcccaatttccccctgcagctcctcgggaagaaggaggagcagacgtgctcgacgtcgcagactgccggggcgggcggcggcggcgtcgtgggcgcgaatgggtcggcggcggcggcgccgccgaagcggacgtcgacgaaggaccggcacacgaaggtggacgggcgggggcggcgcatccggatgccggcgatctgcgccgcgcgggtgttccagctgacgcgggagctcgggcacaagaccgacggcgagaccatcgagtggctgctgcagcaggcggagccggcggtgatcgcggcgaccgggacgggcaccatcccggccaacttcacctccctcaacatctccctccgctcctccggctcgtcgctctccatcccttctcacctccgccttgccggcttggctggccctcgcttcggcggcggcgcgcgggcggcggacgcgtgggaccgcgtcgtcggcctcgggttcggcggtgcggccgacgccccgtcctccgccacctcctcctcctcgtcgccgcttctgctgagcttccactccggtagcgtcggccttgacgtgtcgccgccgtcggcgtcgacctccccggccgccgccgacctctcccggaagcggcggtgggagcaagaaatgcagcagcagcagcagtaccagcagcagatggccgggtacacgcagagccaaattcctgcgggcacggtgtggatggtgccgagcagcaacgcgcaggccgccggtggcggcgctccgccgggaggcggcggcgagtcgatttggacgttcccgcagtcagggagcggcggcggcggcggcgcggcgacagtgtaccgtggcgtgccaagcggactacatttcatgaacttcccggcgacaccaatggcgctgctccccggcgggcagcagctcggcctcgccggcgccggcgggggtggcgaggggcacccggggatcctcgccgcgctcaatgcctaccgcgcacaggccgcgcagccggacgccggcgcggcggcgcagaatggagcgcaaggctcaagtcagcatcgtcagcatcagcatcacggcggcggcggcggcggcggcgacgagcggcatgagagcatgagcgccagcgactcgtagggcttccgttccgtccggtgtgtgccccgcattttctctggttttgatctgttcttctttaattttctccgtttttgtttgagttgttgtctactaatgtctatggctttcttcctcgacccatcatatattcatgcaaggatcatacagttgcattccatgccatctgcataaaaatatcttattgtgtgctcttaagaatggatcaccaatttcaccatgataaagctcttgcaagttgcaagtggtagggtttggttagaatgctagagaaccttttttttaaaaaaaatgaaaatgagaatgctagagaacctagaatcattaatcaataaatcattgctttggtatattgatcgatcgatgaggattatccaccaactaatcctcatccaatgcatgcatgcaagctagctagcatccattcgatctgccactct

SEQ ID NO: 2 - DNA, 1164 bp, Artificial Sequence
  Synthesized
atggatgtcaccggagacggcggaggaggagggcaacggcccaatttccccctgcagctcctcgggaagaaggaggagcagacgtgctcgacgtcgcagactgccggggcgggcggcggcggcgtcgtgggcgcgaatgggtcggcggcggcggcgccgccgaagcggacgtcgacgaaggaccggcacacgaaggtggacgggcgggggcggcgcatccggatgccggcgatctgcgccgcgcgggtgttccagctgacgcgggagctcgggcacaagaccgacggcgagaccatcgagtggctgctgcagcaggcggagccggcggtgatcgcggcgaccgggacgggcaccatcccggccaacttcacctccctcaacatctccctccgctcctccggctcgtcgctctccatcccttctcacctccgccttgccggcttggctggccctcgcttcggcggcggcgcgcgggcggcggacgcgtgggaccgcgtcgtcggcctcgggttcggcggtgcggccgacgccccgtcctccgccacctcctcctcctcgtcgccgcttctgctgagcttccactccggtagcgtcggccttgacgtgtcgccgccgtcggcgtcgacctccccggccgccgccgacctctcccggaagcggcggtgggagcaagaaatgcagcagcagcagcagtaccagcagcagatggccgggtacacgcagagccaaattcctgcgggcacggtgtggatggtgccgagcagcaacgcgcaggccgccggtggcggcgctccgccgggaggcggcggcgagtcgatttggacgttcccgcagtcagggagcggcggcggcggcggcgcggcgacagtgtaccgtggcgtgccaagcggactacatttcatgaacttcccggcgacaccaatggcgctgctccccggcgggcagcagctcggcctcgccggcgccggcgggggtggcgaggggcacccggggatcctcgccgcgctcaatgcctaccgcgcacaggccgcgcagccggacgccggcgcggcggcgcagaatggagcgcaaggctcaagtcagcatcgtcagcatcagcatcacggcggcggcggcggcggcggcgacgagcggcatgagagcatgagcgccagcgactcgtag

SEQ ID NO: 3 - protein, 387 aa, Artificial Sequence
  Synthesized
MDVTGDGGGGGQRPNFPLQLLGKKEEQTCSTSQTAGAGGGGVVGANGSAAAAPPKRTSTKDRHTKVDGRGRRIRMPAICAARVFQLTRELGHKTDGETIEWLLQQAEPAVIAATGTGTIPANFTSLNISLRSSGSSLSIPSHLRLAGLAGPRFGGGARAADAWDRVVGLGFGGAADAPSSATSSSSSPLLLSFHSGSVGLDVSPPSASTSPAAADLSRKRRWEQEMQQQQQYQQQMAGYTQSQIPAGTVWMVPSSNAQAAGGGAPPGGGGESIWTFPQSGSGGGGGAATVYRGVPSGLHFMNFPATPMALLPGGQQLGLAGAGGGGEGHPGILAALNAYRAQAAQPDAGAAAQNGAQGSSQHRQHQHHGGGGGGGDERHESMSASDS

SEQ ID NO: 4 - DNA, 20 bp, Artificial Sequence
  Synthesized
agagtagccatggatgtcac

SEQ ID NO: 5 - DNA, 20 bp, Artificial Sequence
  Synthesized
gagctcgggcacaagaccga

SEQ ID NO: 6 - protein, 5 aa, Artificial Sequence
  Synthesized
RRRRR

SEQ ID NO: 7 - protein, 6 aa, Artificial Sequence
  Synthesized
HHHHHH

SEQ ID NO: 8 - protein, 8 aa, Artificial Sequence
  Synthesized
DYKDDDDK

SEQ ID NO: 9 - protein, 8 aa, Artificial Sequence
  Synthesized
WSHPQFEK

SEQ ID NO: 10 - protein, 10 aa, Artificial Sequence
  Synthesized
EQKLISEEDL

SEQ ID NO: 11 - protein, 9 aa, Artificial Sequence
  Synthesized
YPYDVPDYA

SEQ ID NO: 12 - DNA, 36 bp, Artificial Sequence
  Synthesized
cggtacccggggatccatctatgtcgagaggtgcgg

SEQ ID NO: 13 - DNA, 36 bp, Artificial Sequence
  Synthesized
ggccagtgccaagcttagagtggcagatcgaatgga

SEQ ID NO: 14 - DNA, 21 bp, Artificial Sequence
  Synthesized
tccggccgcttgggtggagag

SEQ ID NO: 15 - DNA, 21 bp, Artificial Sequence
  Synthesized
ctggcgcgagcccctgatgct

SEQ ID NO: 16 - DNA, 18 bp, Artificial Sequence
  Synthesized
gacagtgtaccgtggcgt

SEQ ID NO: 17 - DNA, 20 bp, Artificial Sequence
  Synthesized
cgccgggaagttcatgaaat

SEQ ID NO: 18 - DNA, 20 bp, Artificial Sequence
  Synthesized
accattggtgctgagcgttt

SEQ ID NO: 19 - DNA, 21 bp, Artificial Sequence
  Synthesized
cgcagcttccattcctatgaa

SEQ ID NO: 20 - DNA, 23 bp, Artificial Sequence
  Synthesized
ggcagagtagccatggatgtcac

SEQ ID NO: 21 - DNA, 24 bp, Artificial Sequence
  Synthesized
aaacgtgacatccatggctactct

SEQ ID NO: 22 - DNA, 23 bp, Artificial Sequence
  Synthesized
gccgagctcgggcacaagaccga

SEQ ID NO: 23 - DNA, 23 bp, Artificial Sequence
  Synthesized
aaactcggtcttgtgcccgagct

SEQ ID NO: 24 - DNA, 20 bp, Artificial Sequence
  Synthesized
tctttctagctctaccggcg

SEQ ID NO: 25 - DNA, 20 bp, Artificial Sequence
  Synthesized
cgccgggaagttcatgaaat

(19) United States
(12) Patent Application Publication
(10) Pub. No.: US 2023/0365634 A1
(43) Pub. Date: Nov. 16, 2023

(54) PROTEIN REGULATING NITROGEN-USE EFFICIENCY AND YIELD OF PLANT AND USE THEREOF

(71) Applicant: INSTITUTE OF GENETICS AND DEVELOPMENTAL BIOLOGY, CHINESE ACADEMY OF SCIENCES, Beijing (CN)

(72) Inventors: Chengcai CHU, Beijing (CN); Yongqiang LIU, Beijing (CN); Bin HU, Beijing (CN); Hongru WANG, Beijing (CN)

(21) Appl. No.: 18/245,282

(22) PCT Filed: Sep. 14, 2021
(86) PCT No.: PCT/CN2021/118140

§ 371 Date: Mar. 14, 2023

(30) Foreign Application Priority Data
Sep. 15, 2020 (CN) 202010965332.9

(51) Int. Cl. C07K 14/415; C12N 15/82; C12N 15/11; A01H 6/46

(52) U.S. Cl. CPC C07K 14/415; C12N 15/8261; C12N 15/8243; C12N 15/8218; C12N 15/11; A01H 6/46; C12N 2310/20

(57) ABSTRACT

The present invention discloses a protein regulating nitrogen-use efficiency and yield of a plant and the use thereof. The present invention obtains a transgenic OsTCP19 rice and a rice OsTCP19 mutant by means of a transgenic technique and a CRISPR-Cas9 technique, respectively. Experiments have shown that compared with wild-type rice, the transgenic OsTCP19 rice has a reduced tiller number and a decreased nitrogen response ability, whereas compared with the wild-type rice, the rice OsTCP19 mutant has an increased tiller number. This indicates that the OsTCP19 protein has the function of regulating the plant yield and nitrogen-use efficiency, thus laying the foundation for cultivating varieties with a high yield and a high nitrogen-use efficiency.

### RELATED APPLICATIONS

The present application is a U.S. National Phase of International

Application Number PCT/CN2021/118140 filed Sep. 14, 2021, and claims priority to Chinese Application Number 202010965332.9 filed Sep. 15, 2020.

### INCORPORATION BY REFERENCE

The sequence listing provided in the file entitled C6351-098_SQL, which is an ASCII text file that was created on Mar. 14, 2023, and which comprises 14,015 bytes, is hereby incorporated by reference in its entirety.

### FIELD OF THE INVENTION

The present invention belongs to the field of biotechnology, and specifically relates to a protein regulating nitrogen-use efficiency and yield of a plant and use thereof.

### BACKGROUND OF THE INVENTION

Nitrogen is the mineral nutrient element most required by plants. In agricultural production, the continuous input of chemical fertilizers, mainly nitrogen, has brought a significant increase in grain yield. However, the excessive application of nitrogen fertilizer leads to a large amount of nitrogen fertilizer that is not absorbed by crops into the air, water or remains in the soil, causing air pollution, surface water eutrophication and soil acidification, causing serious environmental damage. In addition, as the world population continues to grow, the demand for food will continue to increase in the future. Therefore, in order to ensure the sustainable development of agriculture, to achieve the breeding goal of “weight loss and increase efficiency” and to improve crop nitrogen-use efficiency (NUE) is critical. Rice is a staple food crop widely grown in the world, and its nitrogen fertilizer application amount is far more than that of other crops. Identifying and utilizing NUE-related genes in rice is of great significance for agricultural production.

NUE is a complex agronomic trait affected by multiple genetic and environmental factors, involving nitrogen uptake, transport, assimilation, reuse, and subsequent growth and development processes. It is difficult to phenotype it with a single indicator. In view of this, the related functional genes have been difficult to clone and identify by traditional genetic methods, which greatly limits the development of nitrogen-efficient breeding.

NUE can be defined as the yield of crops with fixed nitrogen application per unit area, and the yield of rice is determined by the three factors of tiller number, grain number per panicle and thousand-grain weight. Therefore, it is more practical to carry out cloning of NUE-related gene loci using the N response ability of these yield elements (which can also be interpreted as the increase ratio under low nitrogen (LN) to high nitrogen (HN), i.e., (HN-LN)/LN) as the relative phenotypic value of NUE.

### SUMMARY OF THE INVENTION

The first object of the present invention is to provide a new use of an OsTCP19 protein.

The present invention provides the use of an OsTCP19 protein in regulating tillering of a plant and/or yield and/or quality and/or nitrogen-use efficiency and/or nitrogen response ability;

the OsTCP19 protein is any one of the following proteins shown in A1) or A2) or A3) or A4):
A1) a protein consisting of an amino acid sequence shown in SEQ ID NO: 3 in the sequence listing;
A2) a fusion protein obtained by connecting a tag to the N-terminal or/and C-terminal of the protein shown in SEQ ID NO: 3 in the sequence listing;
A3) a protein having a same function as the amino acid sequence shown in SEQ ID NO: 3 in the sequence listing after substitution and/or deletion and/or addition of one or several amino acid residues;
A4) a protein having more than 99%, more than 95%, more than 90%, more than 85% or more than 80% homology with any of the amino acid sequence defined in A1)-A3) and having a same function.

Herein, SEQ ID NO: 3 in the sequence listing consists of 387 amino acid residues.

The labels are specifically shown in Table 1.

TABLE 1
Sequence of tags

| Label | Residues | Sequence |
|---|---|---|
| Poly-Arg | 5-6 | RRRRR |
| | (usually 5) | (SEQ ID NO: 6) |
| Poly-His | 2-10 | HHHHHH |
| | (usually 6) | (SEQ ID NO: 7) |
| FLAG | 8 | DYKDDDDK |
| | | (SEQ ID NO: 8) |
| Strep-tag II | 8 | WSHPQFEK |
| | | (SEQ ID NO: 9) |
| c-myc | 10 | EQKLISEEDL |
| | | (SEQ ID NO: 10) |
| HA | 9 | YPYDVPDYA |
| | | (SEQ ID NO: 11) |

Any of the proteins shown in A1)-A4) above can be synthesized artificially or its encoding gene can be synthesized first and then biologically expressed.

The second object of the present invention is to provide a new use of biological materials related to an OsTCP19 protein.

The present invention provides use of biological materials related to the OsTCP19 protein in any of the following B1)-B4):
B1) regulating tillering of a plant and/or yield and/or quality and/or nitrogen-use efficiency and/or nitrogen response ability;
B2) breeding transgenic plants with increased tiller number and/or increased yield and/or improved quality and/or improved nitrogen-use efficiency and/or improved nitrogen response ability;
B3) breeding transgenic plants with reduced tiller number and/or reduced yield and/or decreased quality and/or decreased nitrogen-use efficiency and/or decreased nitrogen response ability;
B4) plant breeding;

The biological materials related to the OsTCP19 protein is any of the following C 1)-C 8):
C1) a nucleic acid molecule encoding an OsTCP19 protein;
C2) an expression cassette containing the nucleic acid molecule of C1);
C3) a recombinant vector containing the nucleic acid molecule of C1);
C4) a recombinant vector containing the expression cassette of C2);
C5) a recombinant microorganism containing the nucleic acid molecule of C1);
C6) a recombinant microorganism containing the expression cassette of C2);
C7) a recombinant microorganism containing the recombinant vector of C3);
C8) a recombinant microorganism containing the recombinant vector of C4).

In the above use, the nucleic acid molecule in C1) is a DNA molecule of the following 1) or 2) or 3) or 4) or 5):
1) a genomic DNA molecule shown in SEQ ID NO: 1 in the sequence listing;
2) a cDNA molecule shown in SEQ ID NO: 2 in the sequence listing;
3) a DNA molecule derived from rice that has more than 98% homology with the DNA sequences defined in 1) or 2) and encodes a protein related to tillering of a plant and/or yield and/or quality and/or nitrogen-use efficiency and/or nitrogen response ability;
4) a DNA molecule that hybridizes to the DNA sequence defined in 1) or 2) under stringent conditions and encodes a protein related to tillering of a plant and/or yield and/or quality and/or nitrogen-use efficiency and/or nitrogen response ability;
5) a DNA molecule that has more than 90% homology with the DNA sequence defined in 1) or 2) and encodes a protein related to tillering of a plant and/or yield and/or quality and/or nitrogen-use efficiency and/or nitrogen response ability.

Those skilled in the art can easily use known methods, such as directed evolution and point mutation methods, to mutate the nucleotide sequence encoding the OsTCP19 protein of the present invention. Those artificially modified nucleotides that have 75% or more identity to the nucleotide sequence encoding the OsTCP19 protein, as long as they encode the OsTCP19 protein and have the same function, are all derived from the nucleotide sequence of the present invention and are equivalent to sequences of the present invention.

The term “identity” as used herein refers to sequence similarity to a native nucleic acid sequence. “Identity” includes a nucleotide sequence having 75% or higher, or 85% or higher, or 90% or higher, or 95% or higher identity to the nucleotide sequence of the protein composed of the amino acid sequence shown in the encoding SEQ ID NO: 3 of the present invention. Identity can be assessed visually or with computer software. Using computer software, identity between two or more sequences can be expressed as a percentage (%), which can be used to evaluate the identity between related sequences.

The above 90% or more homology can be 91%, 92%, 93%, 94%, 95%, 96%, 97%, 98% or 99% or more homology.

In the above use, the stringent conditions are hybridisation and washing of the membrane twice at 68° C. for 5 min each time in a solution of 2×SSC, 0.1% SDS, also hybridisation and washing of the membrane twice at 68° C. for 15 min each time in a solution of 2×SSC, 0.1% SDS; or, hybridisation and washing of the membrane at 65° C. in a solution of 0.1×SSPE (or 0.1×SSC), 0.1% SDS.

In the above use, the vector can be a plasmid, cosmid, phage or viral vector.

The recombinant vector is a recombinant vector obtained by inserting the above nucleic acid molecule into an expression vector to express the above protein. When using the nucleic acid molecule to construct a recombinant vector, any enhanced, constitutive, tissue-specific or inducible promoter can be added before its transcription initiation nucleotide, and they can be used alone or in combination with other plant promoters; In addition, when using the nucleic acid molecule to construct a recombinant expression vector, enhancers can also be used, including translation enhancers or transcription enhancers, and these enhancer regions can be ATG initiation codons or adjacent region initiation codons etc., but must be in the same reading frame as the encoding sequence to ensure correct translation of the entire sequence. The sources of the translation control signals and initiation codons are extensive and can be natural or synthetic. The translation initiation region can be from a transcription initiation region or a structural gene. In order to facilitate the identification and screening of transgenic plant cells or plants, the plant expression vectors used can be processed, such as adding genes encoding enzymes or luminescent compounds that can produce color changes that can be expressed in plants (GUS gene, luciferase gene, etc.), antibiotic markers with resistance (gentamicin markers, kanamycin markers, etc.) or anti-chemical reagent marker genes (such as herbicide resistance gene). Considering the safety of the transgenic plants, the transgenic plants can be screened directly by adversity without adding any selectable marker gene. In a specific embodiment of the present invention, the recombinant vector is gOsTCP19. The gOsTCP19 is a recombinant vector obtained by inserting the DNA fragment shown in SEQ ID NO: 1 between the BamHI and HindIII restriction sites of the vector pCAMBIA2300-ocs.

In the above use, the microorganisms can be yeast, bacteria, algae or fungi, such as Agrobacterium tumefaciens. The recombinant microorganism is a microorganism containing the above recombinant vector. In a specific embodiment of the present invention, the recombinant microorganism is Agrobacterium tumefaciens AGL1 containing the above recombinant vector.

In the above use, the regulation of plant yield is reflected in the regulation of plant tiller number; the regulation of plant nitrogen-use efficiency is reflected in the regulation of plant nitrogen response ability. The regulation of tillering of a plant and/or yield and/or quality and/or nitrogen-use efficiency and/or nitrogen response ability is specifically embodied in: when the content and/or activity of the OsTCP19 protein in the plant is reduced, the plant tiller number and/or yield and/or quality and/or nitrogen-use efficiency and/or nitrogen responsive ability is increased or improved; when the content and/or activity of the OsTCP19 protein in the plant is improved, the plant tiller number and/or yield and/or quality and/or nitrogen-use efficiency and/or nitrogen response ability is reduced or decreased.

The third object of the present invention is to provide a new use of a substance that inhibits the activity of the OsTCP19 protein or a substance that inhibits the expression of the OsTCP19 protein-encoding gene or a substance that knocks out the OsTCP19 protein-encoding gene.

The present invention provides the use of a substance that inhibits the activity of an OsTCP19 protein or a substance that inhibits the expression of an OsTCP19 protein-encoding gene or a substance that knocks out the OsTCP19 protein-encoding gene in cultivating the transgenic plant with increased tiller number and/or increased yield and/or improved quality and/or improved nitrogen-use efficiency and/or improved nitrogen response ability;

Further, the substance is a CRISPR-Cas9 system; the CRISPR-Cas9 system includes a Cas9 protein and a sgRNA, and the sgRNA targets the encoding gene sequence of an OsTCP19 protein or an upstream promoter sequence thereof or a non-encoding region sequence thereof or a downstream regulatory region sequence thereof.

Furthermore, the target sequence of the sgRNA is a DNA molecule shown in SEQ ID NO: 4 and a DNA molecule shown in SEQ ID NO: 5. The CRISPR-Cas9 system is OsTCP19 CRISPR/Cas9 knockout vector. The OsTCP19 CRISPR/Cas9 knockout vector contains two sgRNAs, denoted as sgRNA 1 and sgRNA 2, respectively. The target sequence of the sgRNA 1 is the DNA molecule shown in SEQ ID NO: 4; the target sequence of the sgRNA 2 is the DNA molecule shown in the SEQ ID NO: 5.

The fourth object of the present invention is to provide a method for cultivating a transgenic plant with increased tiller number and/or increased yield and/or improved quality and/or improved nitrogen-use efficiency and/or improved nitrogen response ability.

The method for cultivating a transgenic plant with increased tiller number and/or increased yield and/or improved quality and/or improved nitrogen-use efficiency and/or improved nitrogen response ability provided by the present invention is as follows D1) or D2):
D1) including the following steps: inhibiting the activity of the OsTCP19 protein in the target plant to obtain a transgenic plant with increased tiller number and/or increased yield and/or improved quality and/or improved nitrogen-use efficiency and/or improved nitrogen response ability;
D2) including the following steps: inhibiting the expression of the OsTCP19 protein-encoding gene in the target plant or knocking out the OsTCP19 protein-encoding gene in the target plant to obtain a transgenic plant with increased tiller number and/or increased yield and/or improved quality and/or improved nitrogen-use efficiency and/or improved nitrogen response ability;

Further, the method for knocking out an OsTCP19 protein-encoding gene in a target plant includes a step of introducing the above CRISPR-Cas9 system into the target plant.

Furthermore, the CRISPR-Cas9 system is the above OsTCP19 CRISPR/Cas9 knockout vector.

The fifth object of the present invention is to provide a method for cultivating a transgenic plant with reduced tiller number and/or reduced yield and/or decreased quality and/or decreased nitrogen-use efficiency and/or decreased nitrogen response ability.

The method for cultivating a transgenic plant with reduced tiller number and/or reduced yield and/or decreased quality and/or decreased nitrogen-use efficiency and/or decreased nitrogen response ability includes the following steps: increasing the activity and/or content of the OsTCP19 protein in the target plant to obtain a transgenic plant with reduced tiller number and/or reduced yield and/or decreased quality and/or decreased nitrogen-use efficiency and/or decreased nitrogen response ability.

Further, the decrease of the nitrogen response ability is specifically reflected in that the tillering nitrogen response value of the transgenic plant is lower than that of the target plants. The calculation formula of tillering nitrogen response value is as follows: (tiller number of high nitrogen treatment group-tiller number of low nitrogen treatment group)/promoted tiller number of low nitrogen treatment. The high nitrogen treatment group is fertilized with 1.5 kg urea per 100 square meters, and the low nitrogen treatment group is fertilized with 0.5 kg urea per 100 square meters.

The method of improving the activity and/or content of the OsTCP19 protein in the target plant is to overexpress the OsTCP19 protein in the target plant. The overexpression method is to introduce the OsTCP19 protein-encoding gene into the target plant.

Furthermore, the OsTCP19 protein-encoding gene is the DNA molecule shown in SEQ ID NO: 1. The OsTCP19 protein-encoding gene is introduced into the target plant through the above recombinant vector gOsTCP19.

Transgenic plants prepared by any of the methods described above also belong to the protection scope of the present invention.

The last object of the present invention is to provide the above CRISPR-Cas9 system.

The above CRISPR-Cas9 system in cultivating transgenic plants or plant breeding with increased tiller number and/or improved yield and/or improved quality and/or improved nitrogen-use efficiency and/or improved nitrogen response ability also belongs to the protection of the present invention scope.

In any of the above uses or methods, the plant can be a monocotyledon or dicotyledon. Further, the monocotyledon can be a gramineous plant. Furthermore, the gramineous plant is specifically rice (such as the rice variety Zhonghua 11).

### BRIEF DESCRIPTION OF THE FIG.

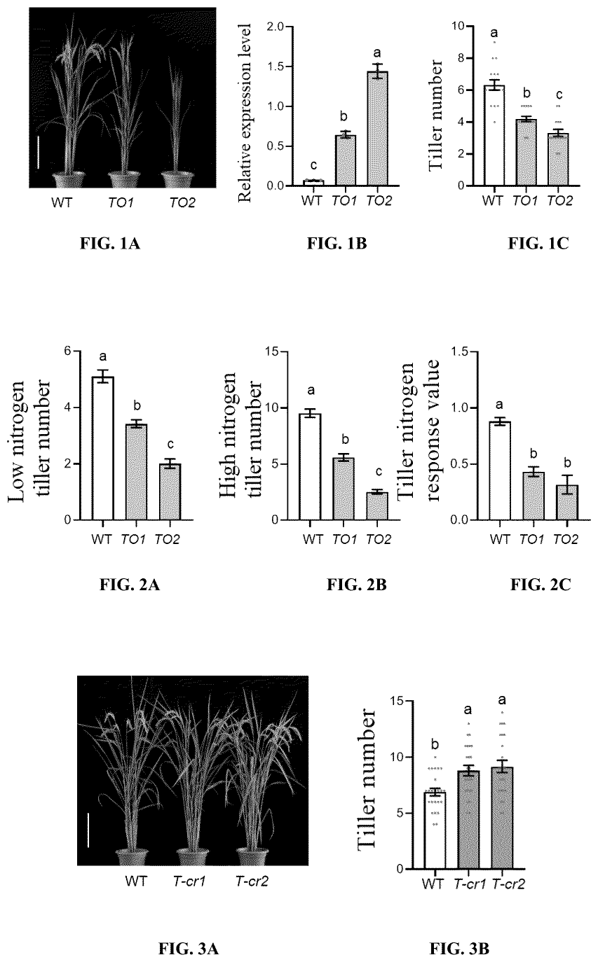

FIGS. 1A-1C show the tiller number in transgenic OsTCP19 rice and wild-type rice. a is the phenotype of transgenic OsTCP19 rice and wild-type rice. b is the detection result of OsTCP19 expression level in transgenic OsTCP19 rice and wild-type rice. c is the tiller number of transgenic OsTCP19 rice and wild-type rice.

FIGS. 2A-2C show the detection of nitrogen response ability of OsTCP19 transgenic rice and wild-type rice. a is the tiller number of transgenic OsTCP19 rice and wild-type rice under low nitrogen treatment. b is the tiller number of transgenic OsTCP19 rice and wild-type rice under high nitrogen treatment. c is the tillering nitrogen response value of transgenic OsTCP19 rice and wild-type rice.

FIGS. 3A and 3B are the tiller number detection of rice OsTCP19 mutant and wild-type rice. FIG. 3A is the phenotype of rice OsTCP19 mutant and wild-type rice. FIG. 3B is the tiller number of rice OsTCP19 mutant and wild-type rice.

### THE BEST WAY TO PRACTICE THE INVENTION

The following examples facilitate a better understanding of the present invention, but do not limit the present invention. The experimental methods in the following examples are conventional methods unless otherwise specified. The test materials used in the following examples, unless otherwise specified, are purchased from conventional biochemical reagent stores. Quantitative experiments in the following examples are all set up to repeat the experiments three times, and the results are averaged.

Zhonghua 11 is described in the following literature: Ma Y, Liu L, Zhu C, Sun C, Xu B, Fang F, Tang J, Luo A, Cao S, Li G, Qian Q, Xue Y, Chu C (2009) Molecular analysis of rice plants harboring a multi-functional T-DNA tagging system, J. Genet. Genomics 36(5):267-276, the public can obtain it from the Institute of Genetics and Developmental Biology, Chinese Academy of Sciences; the biological material is only used to replicate the related experiments of the present invention and cannot be used for other purposes.

pCAMBIA2300-ocs is described the following literature Wang W, Hu B, Yuan D, Liu Y, Che R, Hu Y, Ou S, Zhang Z, Wang H, Li H, Jiang Z, Zhang Z, Gao X, Qiu Y, Meng X, Liu Y, Bai Y, Liang Y, Wang Y, Zhang L, Li L, Mergen S, Jing H, Li J, and Chu C (2018) Expression of the nitrate transporter OsNRT1.1AlOsNPF6.3 confers high yield and early maturation in rice. Plant Cell,30(3): 638-651; the public can obtain it from the Institute of Genetics and Developmental Biology, Chinese Academy of Sciences; the vector can only be used to repeat the related experiments of the present invention, and cannot be used for other purposes.

The pYLsgRNA-U3, pYLsgRNA-U6a and pYLCRISPR/Cas9P_{ubi}-H vectors are all described in the following literature: Ma X, Zhang Q, Zhu Q, et al. A Robust CRISPR/Cas9 System for Convenient, High-Efficiency Multiplex Genome Editing in Monocot and Dicot Plants. Mol. Plant. 2015;8(8):1274-1284, the public can obtain it from the Institute of Genetics and Developmental Biology, Chinese Academy of Sciences, the vector can only be used to repeat the related experiments of the present invention, and cannot be used for other purposes.

Agrobacterium tumefaciens strain AGL1 is described in the following literature: Wang W, Hu B, Yuan D, Liu Y, Che R, Hu Y, Ou S, Zhang Z, Wang H, Li H, Jiang Z, Zhang Z, Gao X, Qiu Y, Meng X, Liu Y, Bai Y, Liang Y, Wang Y, Zhang L, Li L, Mergen S, Jing H, Li J, and Chu C (2018) Expression of the nitrate transporter OsNRT1.1AlOsNPF6.3 confers high yield and early maturation in rice. Plant Cell, 30(3): 638-651; the public can obtain it from the Institute of Genetics and Developmental Biology, Chinese Academy of Sciences, the vector can only be used to repeat the related experiments of the present invention, and cannot be used for other purposes.

### Example 1. Obtaining of Transgenic OsTCP19 Rice and its Tiller Detection

### 1. Obtaining of Transgenic OsTCP19 Rice

### 1. Construction of Recombinant Expression Vector

### 1) Gene Cloning

Zhonghua 11 (Zhonghua 11, ZH11) genomic DNA was used as a template, and the gOsTCP19-F/gOsTCP19-R primer combination (the gOsTCP19-F/gOsTCP19-R primer combination contained two restriction site adapters of BamHI and HindIII, and vector sequence for subsequent homologous recombination) were amplified by PCR to obtain a DNA fragment with a size of 3844bp, the nucleotide sequence of which was shown as SEQ ID NO: 1 in the sequence listing. Herein, positions 1-1944 in SEQ ID NO: 1 were the OsTCP19 gene promoter, positions 1945-2240 were the OsTCP19 gene 5′UTR sequence, positions 2241-3404 were the OsTCP19 gene encoding region sequence, positions 3405-3654 were the OsTCP19 gene 3′UTR sequence, and positions 3655-3844 were downstream sequences of OsTCP19 gene. The primer sequences were specified as follows:

gOsTCP19-F:
(SEQ ID NO: 12)
5′-CGGTACCCGGGGATCCATCTATGTCGAGAGGTGCGG-3′;
gOSTCP19-R:
(SEQ ID NO: 13)
5′-GGCCAGTGCCAAGCTTAGAGTGGCAGATCGAATGGA-3′.

BamHI restriction site was added to the 5′ end of the gOsTCP19 -F primer sequence, and a HindIII restriction site was added to the 5′ end of the gOsTCP19-R primer sequence(underlined). The sequence in bold in the gOsTCP19-F/gOsTCP19-R primer sequence was the sequence on the vector, which was used for subsequent homologous recombination to construct the vector. The italicized sequence in the gOsTCP19-F primer sequence was the nucleotide sequence at positions 1-20 of SEQ ID NO: 1; the italicized sequence in the gOsTCP19-R primer sequence was the reverse complementary sequence of the nucleotide sequence at positions 3825-3844 of SEQ ID NO: 1.

### 2) Construction of Expression Vector

3844bp DNA fragment containing the full length of the OsTCP19 gene obtained in step 1) was inserted between the BamHI and HindIII restriction sites of the vector pCAMBIA2300-ocs to obtain the recombinant expression vector gOsTCP19.

### 2. Obtaining of Transgenic Rice

The recombinant expression vector gOsTCP19 obtained in step 1 was transformed into the Agrobacterium tumefaciens strain AGL1 by heat shock method, and the recombinant Agrobacterium tumefaciens strain containing gOsTCP19 was obtained by screening. Recombinant Agrobacterium tumefaciens strains containing gOsTCP19 were used to infest the healing tissue of Zhonghua 11. For the specific transformation and screening method, please refer to the literature “Yi Zili, Cao Shouyun, Wang Li, He Sijie, Chu Chengcai, Tang Zuoshun, Zhou Puhua, Tian Wenzhong. Improvement of Transformation Frequency of Rice Mediated by Agrobacterium. Journal of Genetics and Genomics, 2001, 28(4):352-358”; finally, the transgenic rice was obtained, the obtained transgenic rice was T0 generation transgenic rice. The seeds of the T0 generation strain were harvested to obtain the T1 generation transgenic rice.

According to the method for obtaining transgenic rice of the T1 generation, the empty vector pCAMBIA2300-ocs was transformed into Zhonghua 11 to obtain empty vector control plants.

### 3. Identification of Transgenic Rice

### 1) PCR Identification

The genomic DNA of T1 generation transgenic rice obtained in step 2 was extracted. The primers F1 and R1 for NptII gene were used for PCR identification. It was identified that the plants containing Nptll gene (the size of PCR product was about 500bp) were positive transgenic rice, named transgenic OsTCP19 rice. The primer sequences were specified as follows:

F1:
(SEQ ID NO: 14)
5′-TCCGGCCGCTTGGGTGGAGAG-3′;
R1:
(SEQ ID NO: 15)
5′-CTGGCGCGAGCCCCTGATGCT-3′.

### 2) Transcriptional Level Analysis (RNA Expression Level)

All T1 generation positive transgenic rice strains obtained in step 1) and wild-type rice variety Zhonghua 11 were used as experimental materials. The total RNA of each material was extracted and reverse transcribed to obtain cDNA. Furthermore, the obtained cDNA was used as a template for real-time quantitative fluorescence PCR against the OsTCP19 gene to detect the expression level of the OsTCP19 gene at the transcriptional level in each material. The experiment was repeated 3 times, and the results were averaged. OsActinl was used as an internal reference gene. The primer sequences used to detect OsTCP19 gene and OsActinl gene were as follows:

OsTCP19-qF:
(SEQ ID NO: 16)
5′-GACAGTGTACCGTGGCGT-3′;
OsTCP19-qR:
(SEQ ID NO: 17)
5′-CGCCGGGAAGTTCATGAAAT-3′;
OsActin1-F:
(SEQ ID NO: 18)
5′-ACCATTGGTGCTGAGCGTTT-3′;
OsActin1-R:
(SEQ ID NO: 19)
5′-CGCAGCTTCCATTCCTATGAA-3′.

OsTCP19-qF was the nucleotide sequence at positions 864-881 of SEQ ID NO: 2, and OsTCP19-qR was the reverse complementary sequence at positions 896-915 of SEQ ID NO: 2.

Based on the results of real-time quantitative fluorescence PCR detection of OsTCP19 gene expression level in each experimental material, two T1 generation strains with medium expression and high expression were selected and named TO1 and TO2 respectively for follow-up research. The detection results of TO1 and TO2 expression levels were shown in the FIG. 1B (empty vector control was consistent with the phenotype of Zhonghua 11 control, which was omitted in FIG. 1B). Compared with the non-transgenic wild-type rice variety Zhonghua 11, the expression level of OsTCP19 gene in T1 generation transgenic OsTCP19 rice strains TO1 and TO2 was significantly increased at the transcriptional level.

### 2. Tiller Detection of Transgenic OsTCP19 Rice

### 1. Detection of Tiller Number in Transgenic OsTCP19 Rice

Under normal conditions, the tillering statistics were carried out on the T1 generation transgenic OsTCP19 rice strains TO1 and TO2, the Zhonghua 11 control plant and the empty vector control plant during the reproductive growth period, and 16 individual plants were counted for each material.

The results were shown in FIG. 1A and FIG. 1C (the empty vector control was consistent with the phenotype of Zhonghua 11 control, which was omitted in FIG. 1A and FIG. 1C). Compared with the Zhonghua 11 control plants and the empty vector control, the tiller numbers of the T1 generation transgenic OsTCP19 rice strains TO1 and TO2 were significantly reduced. Compared with the control Zhonghua 11, the average tiller number of TO1 decreased from 6.3 to 4.2. Compared with the control Zhonghua 11, the average tiller number of TO2 decreased from 6.3 to 3.3.

### 2. Tillering Nitrogen Response Detection of Transgenic OsTCP19 Rice

Further, under high and low nitrogen conditions (low nitrogen: 50 kg ha⁻¹, high nitrogen: 150 kg ha⁻¹), the tiller number of T1 generation transgenic OsTCP19 rice strains TO1 and TO2, Zhonghua 11 control plants and empty vector control were counted, and the tillering nitrogen response value was calculated. Specific treatment steps: two nitrogen fertilizer gradients (high nitrogen and low nitrogen) were set in the field experiment. Herein, the high nitrogen treatment group was fertilized with 1.5 kg urea per 100 square meters, and the low nitrogen treatment group was fertilized with 0.5 kg urea per 100 square meters. Tillering nitrogen response value=(tiller number of high nitrogen treatment group-tiller number of low nitrogen treatment group)/promoted tiller number of low nitrogen treatment, and 16 individual plants were counted under two conditions for each material.

The result was shown in FIGS. 2A-2C. Compared with the Zhonghua 11 control plants and the empty vector control, the tiller numbers of the T1 generation transgenic OsTCP19 rice strains TO1 and TO2 were significantly reduced under both low and high nitrogen conditions (FIG. 2A and FIG. 2B). Herein, under low nitrogen conditions, compared with the control Zhonghua 11, the average tiller number of TO1 decreased from 5.1 to 3.4, and the average tiller number of TO2 decreased from 5.1 to 2. Under high nitrogen conditions, compared with the control Zhonghua 11, the average tiller number of TO1 decreased from 9.5 to 5.5, and the average tiller number of TO2 decreased from 9.5 to 2.5. Furthermore, compared with the Zhonghua 11 control plants and the empty vector control, the nitrogen response ability of the T1 generation transgenic OsTCP19 rice strains TO1 and TO2 was significantly reduced. Herein, compared with the control Zhonghua 11, the average tiller nitrogen response value of TO1 decreased from 0.8 to 0.4, and the average tiller nitrogen response value of TO2 decreased from 0.8 to 0.3 (FIG. 2C).

### Example 2. Obtaining of Rice OsTCP19 Mutant and Its Tiller Detection

1. Obtaining of rice OsTCP19 Mutant
1. Design of sgRNA

Using SEQ ID NO: 1 as a reference sequence, two pairs of sgRNA primer sequences were designed in the OsTCP19 encoding region, which were OsTCP19-U3F/OsTCP19-U3R and OsTCP19-U6aF/OsTCP19-U6aR, respectively. The primer sequences were specified as follows:

OsTCP19-U3F:
(SEQ ID NO: 20)
5′-ggcAGAGTAGCCATGGATGTCAC-3′;
OsTCP19-U3R:
(SEQ ID NO: 21)
5′-aaacGTGACATCCATGGCTACTCT-3′;
OsTCP19-U6aF:
(SEQ ID NO: 22)
5′-gccGAGCTCGGGCACAAGACCGA-3′;
OsTCP19-U6aR:
(SEQ ID NO: 23)
5′-aaacTCGGTCTTGTGCCCGAGCT-3′.

For the convenience of subsequent connection, the 5′ end of the OsTCP19-U3F primer sequence was added with a ggc linker, the 5′ end of the OsTCP19-U3R primer sequence was added with an aaac linker, the 5′ end of the OsTCP19-U6aF primer sequence was added with a gcc linker, and the 5 ′end of the OsTCP19-U6aR primer sequence was added with an aaac linker.

OsTCP19-U3F was the 2232-2251 nucleotide sequence of SEQ ID NO: 1, and OsTCP19-U3R was the reverse complementary sequence of the 2232-2251 nucleotide sequence of SEQ ID NO: 1. OsTCP19-U6aF was the 2505-2524 nucleotide sequence of SEQ ID NO: 1, and OsTCP19-U3R was the reverse complementary sequence of the 2505-2524 nucleotide sequence of SEQ ID NO: 1.

### 2. Construction of Knockout Vector

1) The primer pair OsTCP19-U3F/OsTCP19-U3R was denatured and annealed to obtain the dimer product of OsTCP19-U3F/R. The primer pair OsTCP19-U6aF/OsTCP19-U6aR was denatured and annealed to obtain the dimer product of OsTCP19-U6aF/R.
2) The dimer product of OsTCP19-U3F/R was ligated into a pYLsgRNA-U3 vector, and the dimer product of OsTCP19-U6aF/R was ligated into a pYLsgRNA-U6a vector to obtain two intermediate vectors respectively. Then according to the steps in reference: Ma X, Zhang Q, Zhu Q, et al. A Robust CRISPR/Cas9 System for Convenient, High-Efficiency Multiplex Genome Editing in Monocot and Dicot Plants. Mol Plant.2015;8(8): 1274-1284, the two intermediate vectors were connected together into the pYLCRISPR/Cas9P_{ubi}-H terminal vector by cutting-and-linking method to obtain a double-target OsTCP19 CRISPR/Cas9 knockout vector. The double-target OsTCP19 CRISPR/Cas9 knockout vector contained two sgRNAs, denoted as sgRNA 1 and sgRNA 2, respectively. The target sequence of sgRNA 1 was AGAGTAGCCATGGATGTCAC (SEQ ID NO: 4). The target sequence of sgRNA2 was GAGCTCGGGCACAAGACCGA (SEQ ID NO: 5).

### 3. Obtaining of Rice OsTCP19 Mutant

The knockout vector OsTCP19-CRISPR obtained in step 2 was transformed into the Agrobacterium tumefaciens strain AGL1 by heat shock method, and the recombinant Agrobacterium tumefaciens strain containing OsTCP19-CRISPR was obtained by screening. Recombinant Agrobacterium tumefaciens strains containing OsTCP19-CRISPR were used to infest the healing tissue of Zhonghua 11. For the specific transformation and screening method, please refer to the literature “Yi Zili, Cao Shouyun, Wang Li, He Sijie, Chu Chengcai, Tang Zuoshun, Zhou Puhua, Tian Wenzhong. Improvement of Transformation Frequency of Rice Mediated by Agrobacterium. Journal of Genetics and Genomics, 2001, 28(4):352-358”, finally, the T0 generation transgenic rice was obtained.

### 4. Identification of Rice OsTCP19 Mutant

T0 generation transgenic rice obtained in step 3 was detected by PCR and sequencing. The specific steps were as follows: Genomic DNA of the TO generation transgenic rice obtained in step 3 was extracted. The primers OsTCP19-CRF and OsTCP19-CRR against the OsTCP19 gene were used for PCR identification, and the PCR products were sequenced to obtain a transgenic strain with a mutation in the OsTCP19 gene. The primer sequences were as follows:

OsTCP19-CRF:
(SEQ ID NO: 24)
5′-TCTTTCTAGCTCTACCGGCG-3′;
OsTCP19-CRR:
(SEQ ID NO: 25)
5′-CGCCGGGAAGTTCATGAAAT-3′.

Herein, OsTCP19-CRF was the 2052-2071 nucleotide sequence of SEQ ID NO: 1, and OsTCP19-CRR was the reverse complementary sequence of the 3136-3155 nucleotide sequence of SEQ ID NO: 1.

It was identified by sequencing that two homozygous strains for the OsTCP19 gene mutation were obtained in the T0 generation transgenic rice, and they were named as the rice OsTCP19 mutant strain T-cr1 and the rice OsTCP19 mutant strain T-cr2 respectively.

Compared to the genomic DNA of Zhonghua 11 in wild-type rice, the TO generation rice OsTCP19 mutant strain T-cr1 differs only in that a one-base deletion mutation occurs on both chromosomes in the gene encoding the OsTCP19 protein, which was located at position 2521 of SEQ ID NO: 1, 15 resulting in loss or diminished function of OsTCP19.

Compared to the genomic DNA of Zhonghua 11 in wild-type rice, the TO generation rice OsTCP19 mutant strain T-cr2 differs only in that a 1-base insertion mutation and a 204-base deletion mutation in both chromosomes in the gene encoding the OsTCP19 protein, which has a base insertion positioned between positions 2248 and 2249 of SEQ ID NO: 1 and a deletion mutation located at positions 2319-2522 of SEQ ID NO: 1, resulting in loss or diminished function of OsTCP19.

The seeds of T0 generation rice OsTCP19 mutant strains T-cr1 and T -cr2 were harvested to obtain T1 generation rice OsTCP19 mutant strains T-cr1 and T -cr2, and were used for the following tiller detection.

### 2. Tiller Detection of Rice OsTCP19 Mutant

Under normal conditions, the tillering statistics were carried out on the T1 generation rice OsTCP19 mutant strains T-cr1 and T -cr2, the Zhonghua 11 control plant during the reproductive growth period, and 24 individual plants were counted for each material.

The results were shown in FIGS. 3A and 3B, compared with the Zhonghua control plants, the tiller numbers in the rice OsTCP19 mutant strains T-cr1 and T-cr2 were significantly increased. Herein, the average number of T-cr1 in rice OsTCP19 mutant strains increased from 6.8 to 8.8, and the average number increased from 6.8 to 9.2.

The above are only preferred implementations of the present invention. It should be pointed out that for those skilled in the art, without departing from the technical principle of the present invention, some improvements and modifications can be made, such as gene editing optimization for the OsTCP19 promoter regulatory sequence or the OsTCP19 gene itself, which should also be considered within the scope of protection of the present invention.

### INDUSTRIAL APPLICATION

The present invention provides an OsTCP19 protein related to plant yield and nitrogen-use efficiency, and obtains transgenic OsTCP19 rice and rice OsTCP19 mutants through transgenic technology and CRISPR-Cas9 technology, respectively. Experiments showed that compared with wild-type rice, the tiller number in transgenic OsTCP19 rice reduced, and the nitrogen response ability decreased, while compared with wild-type rice, the tiller number in rice OsTCP19 mutant increased. It shows that OsTCP19 protein has the function of regulating plant yield and nitrogen-use efficiency, and lays the foundation for cultivating varieties with high yield and high nitrogen-use efficiency.

1-20. (canceled)

21. Use of an OsTCP19 protein in regulating tillering of a plant and/or yield and/or quality and/or nitrogen-use efficiency and/or nitrogen response ability;
wherein the OsTCP19 protein is any one of the following proteins shown in A1) or A2) or A3) or A4):
A1) a protein consisting of an amino acid sequence shown in sequence 3 in the sequence listing;
A2) a fusion protein obtained by connecting a tag to a N-terminal or/and a C-terminal of the protein shown in sequence 3 in the sequence listing;
A3) a protein having a same function as the amino acid sequence shown in sequence 3 in the sequence listing after substitution and/or deletion and/or addition of one or several amino acid residues;
A4) a protein having more than 99%, more than 95%, more than 90%, more than 85% or more than 80% homology with any of the amino acid sequence defined in A1)-A3) and having a same function.

22. The use according to claim 21, wherein the plant is M1) or M2) or M3):
M1) monocotyledons or dicotyledons;
M2) gramineous plants;
M3) rice.

23. Use of biological materials related to an OsTCP19 protein in any of the following B1)-B4):
B1) regulating tillering of a plant and/or yield and/or quality and/or nitrogen-use efficiency and/or nitrogen response ability;
B2) breeding transgenic plants with increased tiller number and/or increased yield and/or improved quality and/or improved nitrogen-use efficiency and/or improved nitrogen response ability;
B3) breeding transgenic plants with reduced tiller number and/or reduced yield and/or decreased quality and/or decreased nitrogen-use efficiency and/or decreased nitrogen response ability;
B4) plant breeding;
wherein the biological materials related to the OsTCP19 protein is any of the following C1)-C8):
C1) a nucleic acid molecule encoding the OsTCP19 protein;
C2) an expression cassette containing the nucleic acid molecule of C1);
C3) a recombinant vector containing the nucleic acid molecule of C1);
C4) a recombinant vector containing the expression cassette of C2);
C5) a recombinant microorganism containing the nucleic acid molecule of C1);
C6) a recombinant microorganism containing the expression cassette of C2);
C7) a recombinant microorganism containing the recombinant vector of C3);
C8) a recombinant microorganism containing the recombinant vector of C4);
the OsTCP19 protein is any one of the following proteins shown in A1) or A2) or A3) or A4):
A1) a protein consisting of an amino acid sequence shown in sequence 3 in the sequence listing;
A2) a fusion protein obtained by connecting a tag to the N-terminal or/and C-terminal of the protein shown in sequence 3 in the sequence listing;
A3) a protein having a same function as the amino acid sequence shown in sequence 3 in the sequence listing after substitution and/or deletion and/or addition of one or several amino acid residues;
A4) a protein having more than 99%, more than 95%, more than 90%, more than 85% or more than 80% homology with any of the amino acid sequence defined in A1)-A3) and having a same function.

24. The use according to claim 23, wherein the nucleic acid molecule in C1) is a DNA molecule of the following 1) or 2) or 3) or 4) or 5):
1) a genomic DNA molecule shown in sequence 1 in the sequence listing;
2) a cDNA molecule shown in sequence 2 in the sequence listing;
3) a DNA molecule derived from rice that has more than 98% homology with the DNA sequences defined in 1) or 2) and encodes a protein related to plant tiller number and/or yield and/or quality and/or nitrogen-use efficiency and/or nitrogen response ability;
4) a DNA molecule that hybridizes to the DNA sequence defined in 1) or 2) under stringent conditions and encodes a protein related to plant tiller number and/or yield and/or quality and/or nitrogen-use efficiency and/or nitrogen response ability;
5) a DNA molecule that has more than 90% homology with the DNA sequence defined in 1) or 2) and encodes a protein related to plant tiller number and/or yield and/or quality and/or nitrogen-use efficiency and/or nitrogen response ability.

25. The use according to claim 23, wherein the plant is M1) or M2) or M3):
M1) monocotyledons or dicotyledons;
M2) gramineous plants;
M3) rice.

26. Any of the following uses:
N1) use of a substance that inhibits the activity of an OsTCP19 protein in cultivating transgenic plants with increased tiller number and/or increased yield and/or improved quality and/or improved nitrogen-use efficiency and/or improved nitrogen response ability;
N2) use of a substance that inhibits the expression of an OsTCP19 protein-encoding gene or a substance that knocks out the OsTCP19 protein-encoding gene in breeding transgenic plants with increased tiller number and/or increased yield and/or improved quality and/or improved nitrogen-use efficiency and/or improved nitrogen response ability;
wherein the OsTCP19 protein is any one of the following proteins shown in A1) or A2) or A3) or A4):
A1) a protein consisting of an amino acid sequence shown in sequence 3 in the sequence listing;
A2) a fusion protein obtained by connecting a tag to the N-terminal or/and C-terminal of the protein shown in sequence 3 in the sequence listing;
A3) a protein having a same function as the amino acid sequence shown in sequence 3 in the sequence listing after substitution and/or deletion and/or addition of one or several amino acid residues;
A4) a protein having more than 99%, more than 95%, more than 90%, more than 85% or more than 80% homology with any of the amino acid sequence defined in A1)-A3) and having a same function.

27. The use according to claim 26, wherein the substance is a CRISPR-Cas9 system; the CRISPR-Cas9 system comprises a Cas9 protein and a sgRNA, and the sgRNA targets an encoding gene sequence of the OsTCP19 protein or an upstream promoter sequence thereof or a non-encoding region sequence thereof or a downstream regulatory region sequence thereof.

28. The use according to claim 27, wherein the target sequence of the sgRNA is a DNA molecule shown in sequence 4 and a DNA molecule shown in sequence 5.

29. The use according to claim 26, wherein the plant is M1) or M2) or M3):
M1) monocotyledons or dicotyledons;
M2) gramineous plants;
M3) rice.

30. A method for cultivating a transgenic plant with increased tiller number and/or increased yield and/or improved quality and/or improved nitrogen-use efficiency and/or improved nitrogen response ability, which is the following D1) or D2):
D1) comprising the following steps: inhibiting the activity of an OsTCP19 protein in a target plant to obtain the transgenic plant with increased tiller number and/or increased yield and/or improved quality and/or improved nitrogen-use efficiency and/or improved nitrogen response ability;
D2) comprising the following steps: inhibiting the expression of an OsTCP19 protein-encoding gene in the target plant or knocking out the OsTCP19 protein-encoding gene in the target plant to obtain the transgenic plant with increased tiller number and/or increased yield and/or improved quality and/or improved nitrogen-use efficiency and/or improved nitrogen response ability;
wherein the OsTCP19 protein is any one of the following proteins shown in A1) or A2) or A3) or A4):
A1) a protein consisting of an amino acid sequence shown in sequence 3 in the sequence listing;
A2) a fusion protein obtained by connecting a tag to the N-terminal or/and C-terminal of the protein shown in sequence 3 in the sequence listing;
A3) a protein having a same function as the amino acid sequence shown in sequence 3 in the sequence listing after substitution and/or deletion and/or addition of one or several amino acid residues;
A4) a protein having more than 99%, more than 95%, more than 90%, more than 85% or more than 80% homology with any of the amino acid sequence defined in A1)-A3) and having a same function.

31. The method according to claim 30, wherein the method for knocking out the OsTCP19 protein-encoding gene in the target plant comprises a step of introducing a CRISPR-Cas9 system into the target plant; the CRISPR-Cas9 system comprises a Cas9 protein and a sgRNA, and the sgRNA targets an encoding gene sequence of the OsTCP19 protein or an upstream promoter sequence thereof or a non-encoding region sequence thereof or a downstream regulatory region sequence thereof.

32. The method according to claim 31, wherein the target sequence of the sgRNA is a DNA molecule shown in sequence 4 and a DNA molecule shown in sequence 5.

33. The method according to claim 30, wherein the plant is M1) or M2) or M3):
M1) monocotyledons or dicotyledons;
M2) gramineous plants;
M3) rice.

34. A method for cultivating a transgenic plant with reduced tiller number and/or reduced yield and/or decreased quality and/or decreased nitrogen-use efficiency and/or decreased nitrogen response ability, comprising the following steps: increasing the activity and/or content of an OsTCP19 protein in a target plant to obtain the transgenic plant with reduced tiller number and/or reduced yield and/or decreased quality and/or decreased nitrogen-use efficiency and/or decreased nitrogen response ability;
the OsTCP19 protein is any one of the following proteins shown in A1) or A2) or A3) or A4):
A1) a protein consisting of an amino acid sequence shown in sequence 3 in the sequence listing;
A2) a fusion protein obtained by connecting a tag to the N-terminal or/and C-terminal of the protein shown in sequence 3 in the sequence listing;
A3) a protein having a same function as the amino acid sequence shown in sequence 3 in the sequence listing after substitution and/or deletion and/or addition of one or several amino acid residues;
A4) a protein having more than 99%, more than 95%, more than 90%, more than 85% or more than 80% homology with any of the amino acid sequence defined in A1)-A3) and having a same function.

35. The method according to claim 34, wherein, the plant is M1) or M2) or M3):
M1) monocotyledons or dicotyledons;
M2) gramineous plants;
M3) rice.

36. A transgenic plant prepared by the method of claim 30.

37. A transgenic plant prepared by the method of claim 34.

38. A CRISPR-Cas9 system comprising a Cas9 protein and a sgRNA, wherein a target sequence of the sgRNA is a DNA molecule shown in sequence 4 and a DNA molecule shown in sequence 5.

39. Use of the CRISPR-Cas9 system according to claim 38 in cultivating a transgenic plant or plant breeding with increased tiller number and/or increased yield and/or improved quality and/or improved nitrogen-use efficiency and/or improved nitrogen response ability.

40. The method according to claim 39, wherein the plant is M1) or M2) or M3):
M1) monocotyledons or dicotyledons;
M1) gramineous plants;
M3) rice.
